(19) United States
(12) Patent Application Publication
(10) Pub. No.: US 2009/0306485 A1
(43) Pub. Date: Dec. 10, 2009

(54) Wearable Electronic System

(76) Inventor: Jonathan Arnold Bell, Culver City, CA (US)

Correspondence Address: Jonathan Arnold Bell, 5314 South Slauson Avenue, Culver City, CA, 90230, US

(21) Appl. No.: 12/477,148

(22) Filed: Jun. 3, 2009

Related U.S. Application Data
(60) Provisional application No. 61/058,539, filed on Jun. 3, 2008.

(51) Int. Cl. A61B 5/00; A61B 5/04

(52) U.S. Cl. 600/301; 600/388

(57) ABSTRACT

This document describes the design and control of a modular wearable electronic system that integrates an electrical interconnection harness, human body electrode modules, physiological sensor modules, electronic circuit modules, control software, and power supply modules into a single assembly. The design is intended to allow medical sensors and electronic circuits from different manufacturers to be connected into the system with relative ease. This system will enable a platform that can be expanded to incorporate many different kinds of physiological sensors and electronic circuits as and when they become available. It will also allow for different sizes of wearable electronic system to be constructed by simply changing the lengths and shapes of the electrical interconnections between the electrical modules.

### CROSS-REFERENCE TO RELATED APPLICATIONS

Provisional Patent Application No. 61/058,539.

### STATEMENT REGARDING FEDERALLY SPONSORED RESEARCH OR DEVELOPMENT

Not applicable.

### REFERENCE TO SEQUENCE LISTING, A TABLE OR A COMPUTER PROGRAM LISTING COMPACT DISC APPENDIX:

Not applicable.

### BACKGROUND OF THE INVENTION

An early example of a portable wearable electronic system is the battery powered heated sock used for warming feet in old climates. More recent examples have been used for monitoring the personal health of an individual. During the NASA Apollo space missions of the 1960s, a bio-belt consisting of various electronic processing boxes was attached around the waist and used to measure the heart electro-cardiogram (ECG) signals of the astronauts via wire cables attached to skin electrodes positioned across the upper body surface. Highly accurate ECG measurements can require up to 10 separate wire cables connected to the body making the attachment procedure slow and the dangling wire cables cumbersome. To overcome this problem, a relatively new design from NASA Ames Research Center called the lifeguard system experimented with a single flexible plate mounted on the breast area that incorporates multiple electrodes and multiple wiring traces that plug into a central electronic processing box placed over the stomach area. The design concept of grouping the electronic processing and power supply together typically requires a significantly bulky box to house the electronics making it unsuitable for use underneath an item of clothing. For NASA astronauts this is further complicated by the use of a Liquid Cooling and Ventilation Garment (LCVG) used to cool the body during extra vehicular activities (EVAs) that is a skin tight garment covering the entire skin area of the legs, arms, and torso. A comfortable and effective wearable electronic system would appear to require that all electronics be distributed around the body in discrete modules so that each discrete electronic module is low profile. This has the added advantage of distributing electronic circuit mass around the body rather than at a central point. It would also appear that eliminating the ‘spaghetti’ type wiring commonly associated with ECG skin electrode connections is desirable. A combination of body electrodes, physiological sensors, electronic circuits, and power supply, all connected on a common communications bus that transmits and receives analog and digital electrical signals will allow for additional sensors to be added to the system without additional wiring.

Previous authors on the subject of wearable electronics have described systems that may be worn on the wrist, arm, waist, chest, head, and torso and a brief summary of their work is outlined as follows. Righter et al describe a portable, multi-channel ECG data monitor in the form of a wrist band or necklace that uses wire cables to connect body electrodes to a central processor. Stivorich et al describe a system for monitoring health wellness and fitness in the form factor of an armband. Rytky describes a garment and heart rate monitor sensor system in the form factor of a chest band and waistband with similar use of wire cables to connect body electrodes to a central processor. Groff et al describe a wearable vital signs monitor in the form of a chest band that measures ECG signals, respiration rate, oxygen sensing, and temperature. Ryu et al describe a wearable physiological signal detector in the form of a t-shirt with body electrodes placed at various positions. The electrodes appear to use a wireless radio method to transmit detected physiological data to a central processor. Farrell et al describe a physiological status monitoring system in the form of a t-shirt that uses insulated wire cables to circle the body and electrically connect electrodes and sensors with a central processor. DeFusco et al describe a textile based body electrode structure that is woven from conductive yarn and uses a metal stud as means of connecting with external devices. These electrodes may be woven into garments at particular positions for detecting physiological signals. Jayaraman et al describe a garment with an integrated flexible information infrastructure that uses optical fibers and wire cables interwoven to form a garment fabric that incorporates the ability to transmit electrical signals along the woven wire paths. Sackner et al describe systems and methods for ambulatory monitoring of physiological signs that uses wire cables to interconnect medical sensors to a central processor within a vest like form factor. Chmiel et al describe a wireless biometric monitoring system that uses modular physiological sensors connected along a belt like wearable form factor. The function of each module is decided by inserting a pre-programmed circuit board into each of the available modules strung along the belt. Many of these devices are commercially available from companies such as Polar, Bodymedia, Zephyr, Hidalgo, and Vivometrics.

### OBJECTS OF THE INVENTION

1) One object of the present invention is to provide a wearable electronic system that integrates an electrical harness, human body electrodes, biological sensors, electronic circuits, control software, and a battery power supply into a single assembly.

2) a low height profile so that the LCVG does not snag on the monitor system during donning and doffing.

3) electronic circuits distributed around the body to disperse bulk and mass

4) flexible snap-fit interconnects that conform to the human body shape and reduce chafing

5) allows medical sensors from different manufacturers to be plugged into the system with relative ease.

### BRIEF SUMMARY OF THE INVENTION

The basic electrical harness is constructed of physiological sensor modules, processing circuit modules, and body electrode modules, all interconnected with a central micro-controller circuit (MCU) over a digital data-bus. Modules are generally fabricated from a flexible copper/polyimide circuit material using surface mount component technology and will be protected with a semi-rigid molding. The semi-rigid modules will be designed as small parts with rounded edges and corners to avoid chafing. The interconnecting data-bus will be highly flexible and bendable made of multiple stranded-core wires and/or flat copper flex circuit. Polyimide and polyurethane materials may be used to strengthen and waterproof the harness while still allowing for a bendable composite with good garment drape characteristics. Combining the harness with a fabric cloth such as cotton should allow for a comfortable, wearable assembly. Attachment of the harness to the fabric may be achieved using a suitable adhesive.

The analog and digital data-bus is implemented as a means to reduce the number of individual electrical connections required to access all the sensors/circuits. A short example of operation is as follows; once the battery power supply is switched on, the software operating system inside the micro-controller unit (MCU) is activated. The MCU sends a message out along the data-bus that requests a sensor/circuit to transmit its' present reading back to the MCU. Once the data is received within the MCU it may be processed arbitrarily and then sent to a memory storage device located on the data-bus, such as an SD flash memory card for later removal, or transmitted wirelessly to another physical location such as a personal computer.

At present, NASA extravehicular activity (EVA) activities typically require the astronaut to wear a liquid cooling and ventilation garment (LCVG) that covers the arms, legs, and torso areas of the body in an elastic, form fitting manner. This requires that the wearable electronic system lie underneath the cooling garment so that electrodes can be attached directly to the human skin. As a result, the wearable electronic system must be designed to be low profile so that the LCVG does not snag on the monitor system during donning and doffing, and should allow for a snug fit underneath the LCVG so that it does not chafe the wearer. It is also important to consider the type of fabric used in the electronic system and minimize the surface area of fabric used so as not to interfere with the correct operation of the LCVG.

There is also a need for electrodes attached to the skin within the wearable electronic system to be easily replaced after a certain number of hours of use. The proposed design will pioneer a method whereby commercially available off the shelf electrodes may be connected and disconnected directly to the electrical harness of the wearable electronic system using a standard snap-fit connector. Donning and doffing of the wearable electronic system could simplify the correct placement of any required skin electrodes. Because the skin electrodes are integrated into the harness, it is considered that by donning the wearable electronic system in an appropriate manner, the electrodes will automatically be positioned within the correct region of the body.

There are a number of different protocols available for controlling communications along a serial digital data-bus. One of the most common is termed I2C (sometimes pronounced I squared C) and is an acronym for Inter-IC bus. This protocol was developed by the Royal Dutch Philips Company and has been adopted as an industry standard. It is a two-wire bus structure where one wire is used to communicate data and the other wire is for a synchronous clock signal used to latch data into and out of digital devices. Each digital device placed on the bus has it own address number so that it may be addressed uniquely and this address number is usually set by connecting pull-up or pull-down resistors to the leads of the surface mount device package. I2C has 7-bit and 10-bit addressing schemes that allow for more than 100 individually addressable devices on a single bus. The data rate can be as high as 3.4 Mbits/sec. Due to the approximate 8 feet length of the anticipated data-bus, an additional set of resistors and capacitors may be required to dampen any digital signal echoes that may occur. These resistors and capacitors will be placed wherever an I2C device is required on the data-bus. Universal Serial Bus (USB) is another popular protocol commonly used to connect PCs with external devices such as printers. While USB currently allows for higher data rates, the complexity of the protocol software required to correctly operate the devices strung along the bus is potentially an order of magnitude above that required for I2C. As a consequence of this, it is suggested that I2C will simplify design and increase the likelihood of a successful design but does not exclude USB or other types of communications protocol. The initial design will separate the data-bus into individual pieces that interconnect each individual node. Connections between the data-bus and the sensors/circuits will be made using snap-fit electro-mechanical connectors. In addition to the two electrical traces required for I2C communication, a third and fourth trace are required for electrical power and electrical ground respectively.

Electronic circuit modules are the basic electronic components and circuits required to complete the electrical harness and are generally an MCU, for general operation of the system, a removable memory device for storage of the measured health data, a wireless transceiver for communicating with a remote PC, and a power supply battery module. The choice of MCU depends on a number of different factors such as physical size, speed of operation, heat generation, on-board memory (ROM, RAM, and Flash), digital communication ports, etc. The choice of removable memory storage device is likely to be a solid-state flash type device such as SanDisk memory cards. These are commonly used in today's consumer electronic products and may store more than 1 giga-byte of information in a physical size less than 25 mm×25 mm×2 mm. There are many wireless digital communication technologies currently available such as radio based Wifi, Zigbee, and Bluetooth, or optical infra-red, etc. These offer omni-directional communication for radio waves and highly directional communication for optical waves. Radio based technology therefore appears more appropriate for the wearable electronic system. Both Zigbee and Bluetooth offer transmission distances of 10 to 100 feet with relatively low electrical power consumption. This is an important concern when considering battery lifetime.

Electronic devices are typically designed to operate within a lower and upper limit of voltage supplied by the battery, e.g., 5.0V+/−0.5V. It is therefore important that the correct voltage is supplied to all the electronics. Voltage converter devices may be used to step-up or step-down the voltage levels as needed.

A physical switch mechanism used to power the electronic system on and off will also be included. A switch that cannot be accidentally operated, for example when a LCVG is donned on top of the electronic system, is required. A small light emitting diode (LED) is also recommended as a simple means to determine if the unit is switched on or off.

Electrode modules that adhere to the human skin to detect such signals as ECG are available from a number of different commercial suppliers. The ‘Red Dot’ type from the 3M Corporation appears well suited for use in the wearable electronic system. These electrode/adhesive combinations are approximately 30 mm×30 mm in size and come with a standard snap-fit style plug connector. It should also be noted that in the case of ECG electrodes, there are at least three separate electrodes positioned at three different points on the body, e.g., the right arm, left arm, and left leg positions of Einthoven's triangle. The three electrical potentials measured at each of the three electrodes are typically fed into a single electronic circuit by means of three connecting cables attached to the electrodes. This electronic circuit then outputs a waveform representing the ECG signal. This new wearable electronic system design will use individual copper traces on a single flex circuit data-bus to connect each ECG electrode to the electronic processing circuit or alternatively, insulated, stranded wire cables.

The voltage potential detected at each ECG electrode is of a relatively low signal strength (mV range) when compared to the 3V to 5V digital signals likely to pass along the digital data-bus. If the ECG electrical traces are placed next to the data-bus traces it is possible that the ECG signal will be adversely affected by electrical noise. Therefore a separate flex circuit is proposed that only carries ECG and/or other low signal strength analog electrical signals. This ‘ECG-bus’ would lie directly beneath the digital data-bus structure or may be integrated in the same layer as the digital data-bus and would run alternate electrical ground traces on either side of each ECG trace to give additional electrical shielding from noise. Stranded-core wire may offer an alternative method for connecting the ECG electrodes. In this case the plastic insulated wires would follow the same path as the flex circuit bus structure but would likely be less sensitive to noise pick-up. A further method for ECG detection might be to digitize the ECG voltage potential measured at each ECG electrode. This would be achieved by sampling each ECG electrode signal using an analog-to-digital converter (ADC) relative to electrical ground and then transmitting the digitized value along the I2C digital data-bus to the MCU for mathematical processing.

The electrical harness made of the data-bus, sensors, electronic circuits, power supply, and electrodes is not a complete unit ready to be worn. To make a wearable electronic system that is practical to wear, a fabric backing material is suggested. This fabric holds the different components of the electrical harness in position while donning, doffing, and in storage, and also allows for physical features such as straps and fasteners to be readily incorporated. The likely characteristics of any chosen fabric are that it is comfortable next to the skin, washable, non-shrinking, breathable, electro-static free, fire-resistant, lightweight, and does not outgas. Brushed, natural cotton of the type commonly used in T-shirts may be a good candidate. By designing the fabric pattern as shown in FIG. 3, seams across the shoulder area of the astronauts' body that might cause chafing can be avoided. Small openings in the fabric at the electrode positions allow for self adhesive electrodes to be attached and removed. Fabrics that are pre-bonded to a release liner are particularly useful as they can be marked out and cut with high precision on a computer controlled x-y plotter/cutting machine before the release liner is removed. This allows the cut pattern sizes to match the electrical circuit mask designs to a tolerance of less than 1 mm over a 1 m distance. Many different types of natural and manmade fabrics are available with this release liner technology which also allows for accurate, waterproof inkjet printing directly onto the fabric surface.

The basic electrical harness can be described as consisting of various circuit modules and the interconnections between them. Flex interconnections may be strengthened and waterproofed by applying an adhesive backed polyimide material to the copper flex circuit. The circuit nodes may be waterproofed using a polyurethane material. Both circuit nodes and interconnections may be attached to the fabric backing using a pressure sensitive adhesive allowing for the fabric and harness assembly to retain acceptable garment drape characteristics.

### BRIEF DESCRIPTION OF THE SEVERAL VIEWS OF THE DRAWING

FIG. 1 shows examples of wearable electronic health monitors of the past and present.

FIG. 2 shows the proposed design for a wearable electronic system.

FIG. 3 shows the snap-fit structure of the proposed wearable electronic system using modular electronic circuits and sensors interconnected with a flexible data-bus.

FIG. 4 shows two mechanical housing modules, one flat and one curved shaped.

FIG. 5 shows a method of connecting an electrical body sensor (electrode) through a cloth opening into a mechanical housing.

FIG. 6 shows a method for removing an electrical body sensor (electrode) from a mechanical housing.

FIG. 7 shows a method for transferring an electrical body sensor (electrode) signal to one of many different electrical interconnections.

FIG. 8 shows a method for protecting the solder joints and electrical traces of a surface mounted integrated circuit and connector.

FIG. 9 shows a method for constructing the analog and digital data-bus interconnections.

FIG. 10 shows a method for interconnecting circuits and sensors with pre-cut and pre-shaped lengths and curvatures that are used to space the modules and sensors at appropriate distances from one another around the human body.

FIG. 11 shows a method for interconnecting circuits and sensors with spiral-like windings to reduce stress on the electrical interconnections caused by bending and/or twisting.

FIG. 12 shows a method for interconnecting circuits and sensors with serpentine-like shapes and concertina-like shapes to allow for stretching of the interconnects.

FIG. 13 shows a wearable electronic system combined with a cloth-like fabric where openings are created in the cloth garment at strategic locations to allow an electronic circuit and/or sensor module to be accessed without any removal of the cloth-like fabric and allows the electrical data-bus interconnections to be enclosed between the fabric layers.

FIG. 14 shows two different garment designs for donning and doffing over a human torso.

FIG. 15 shows an example of a battery module with dual electro-mechanical connectors.

### DETAILED DESCRIPTION OF THE INVENTION

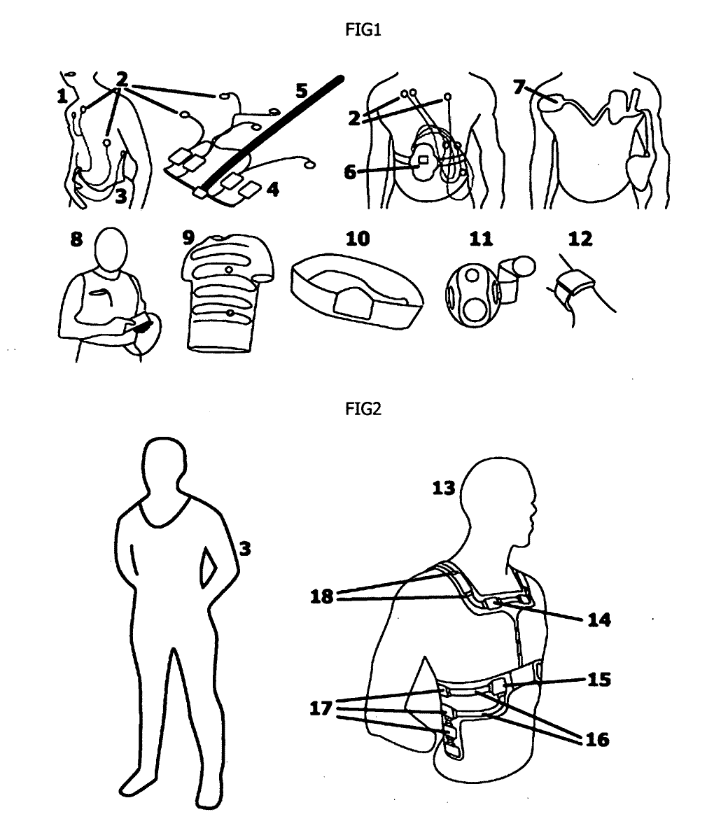

FIG. 1 shows a wearable electronic health 1 monitor worn by NASA astronauts during the Apollo moon missions. A series of electrical body sensors (electrodes) 2 are shown attached to the upper torso. These are worn underneath the tight fitting liquid cooling ventilation garment 3. Also shown are the electronic circuit modules 4 strung around the waist in a belt like fashion and connected through a multi-core electrically conductive cable 5. The electronic circuit modules 4 are condensed into a smaller unit 6 shown attached to the frontal chest area of the Lifeguard wearable electronic health monitor. ECG electrodes 2 are substituted for a partially integrated set of electrodes 7 believed from the Nexan company. The Lifeshirt wearable electronic health monitor from the Vivometrics company is shown 8, a shirt consisting of woven wires and optical fibers from the Sensatex company is shown 9, a waistband from the Zephyr company is shown 10, an armband from the Bodymedia company is shown 11, and a wristband from the Exmocare company is shown 12.

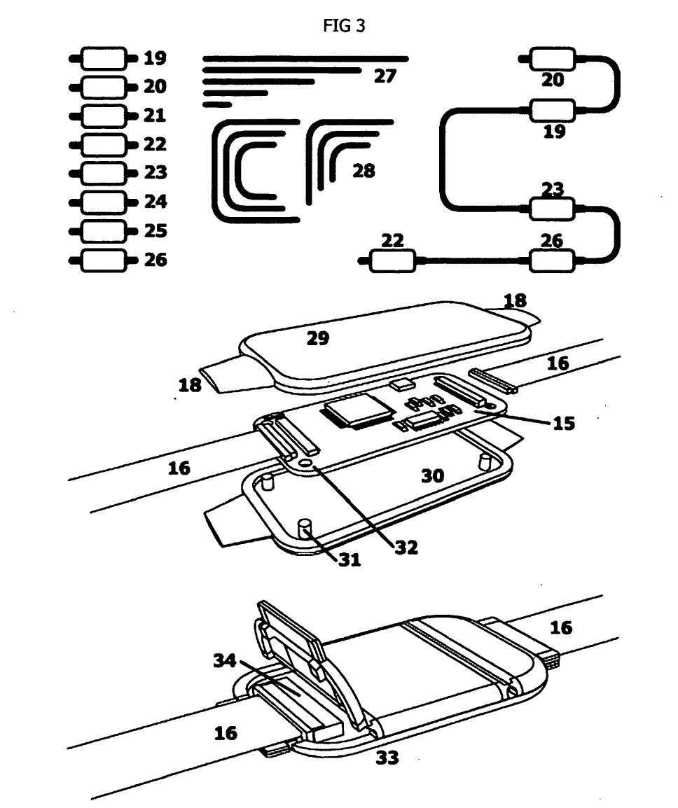

FIG. 2 shows the current liquid cooling ventilation garment used by NASA astronauts 3, and the newly proposed wearable electronic system 13 that partly consists of mechanical housings 14 for containing electronic circuits and sensors 15 connected on a common data-bus structure 16. The electronic circuits and sensors and their associated mechanical housings 17 can be snapped into or out of the system and have strain reliefs 18 at the entrance and exit points of the mechanical housings.

FIG. 3 the shows the snap-fit structure of the proposed wearable electronic system using modular electronic circuits and sensors interconnected with a common data-bus, for example micr-controller module 19, battery module 20, wireless transceiver module 21, optical display module 22, altimeter module 23, gas monitor 24, memory module 25, and thermometer module 26. These may be interconnected with straight sections 27 of common data-bus and/or curved sections 28. An upper clamshell 29 and lower clamshell 30 of a mechanical housing is also shown where common data-bus interconnects 16 are attached to an electronic circuit or sensor 15. The sensor circuit board has holes 32 that mate with posts 31 of the mechanical housing for added strength. Strain reliefs 18 are positioned at the points where the common data-bus interconnects are attached. A hinge design shown 33 allows an opening in the mechanical housing to be revealed giving access to the common data-bus interconnects and their mating electro-mechanical connectors 34.

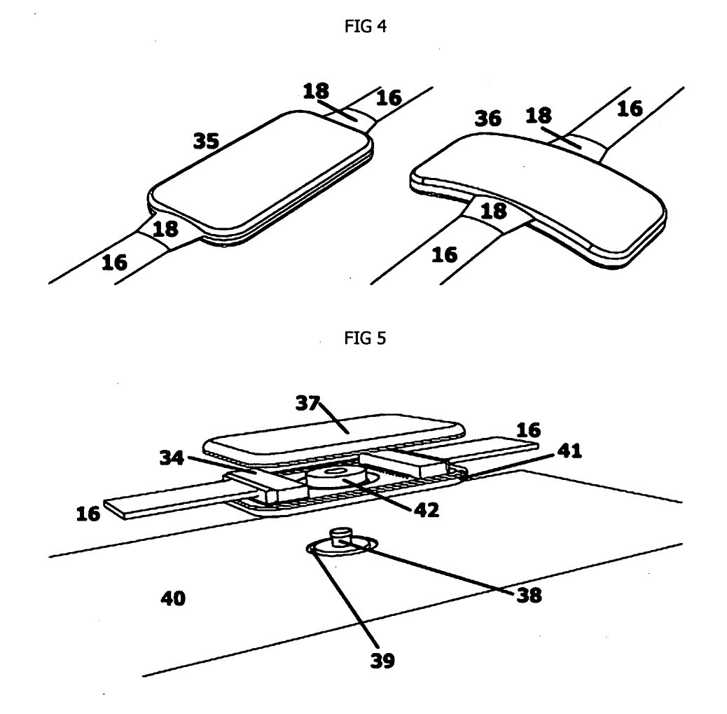

FIG. 4 shows two mechanical housing modules, one flat 35, and one curved shaped 36, with strain reliefs 18 and common data-bus interconnects 16.

FIG. 5 shows a method of connecting an electrical body sensor (electrode) 38 through an opening 39 in the garment cloth 40 through an opening in the underside of the mechanical housing 41 to connect with a retainer mechanism 42 inside the mechanical housing.

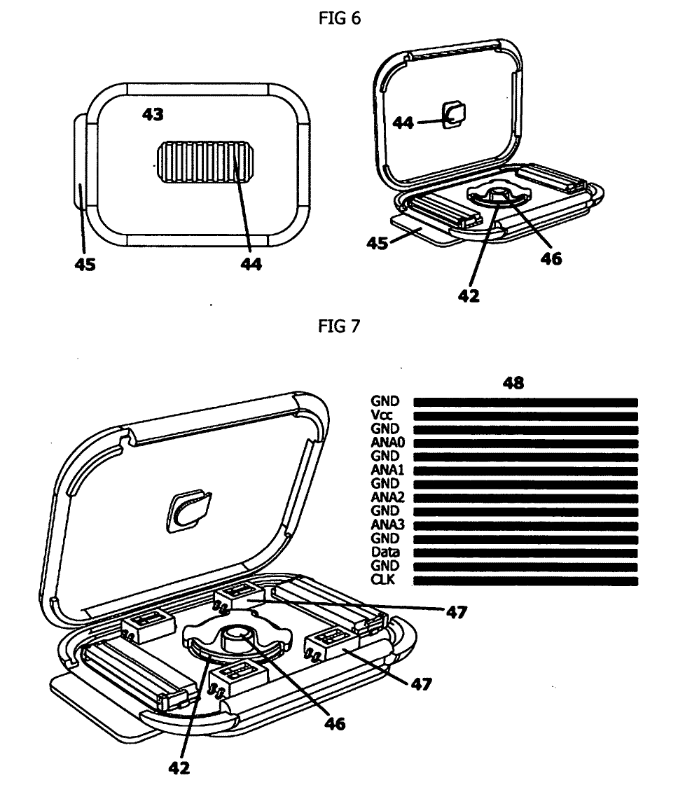

FIG. 6 shows a method for removing an electrical body sensor (electrode) 45 from a mechanical housing 43 and retaining fixture 42 through use of a mechanical fixture 44 that impinges on the electrical body sensor (electrode) through opening 46. The mechanical fixture 44 pushes the electrical body sensor (electrode) 45 out of the retaining fixture 42 through use of a pushing action.

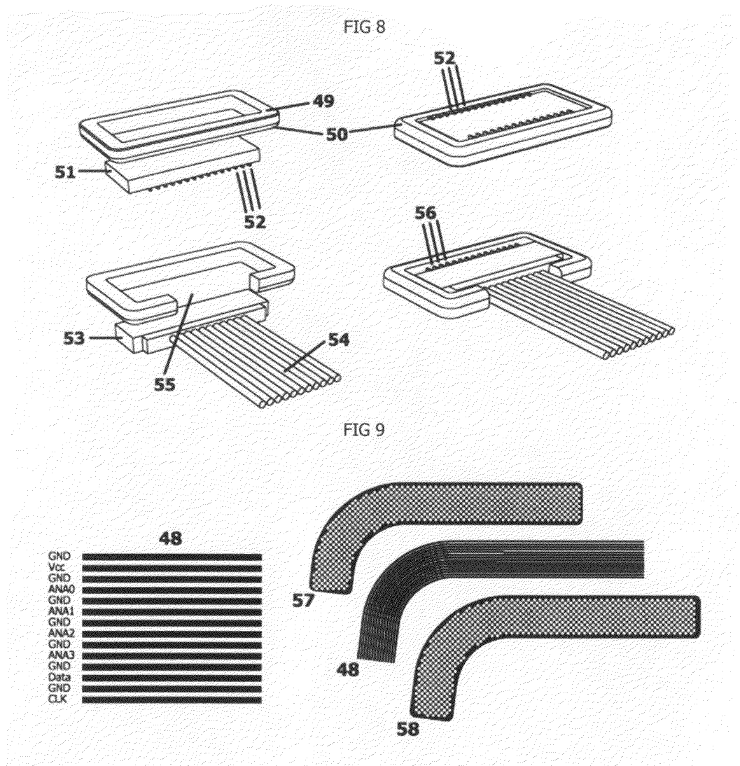

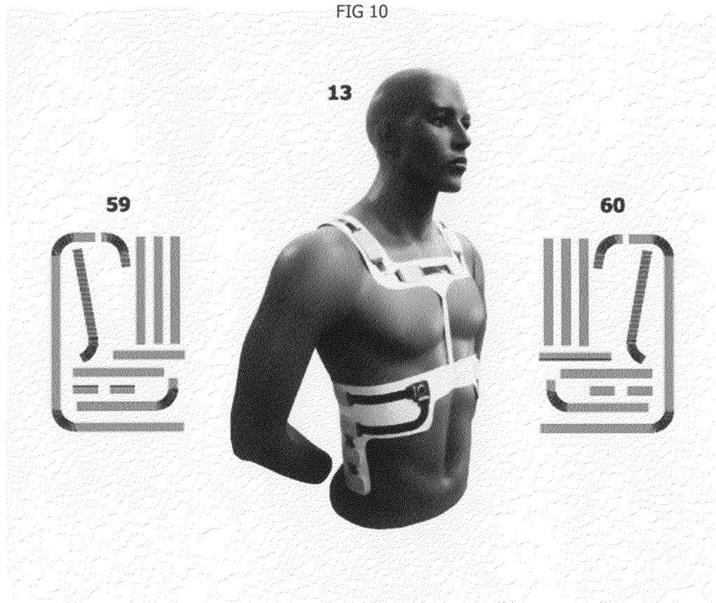

FIG. 7 shows a method whereby an electrical body sensor (electrode) signal connected to retaining fixture 42 is connected to one of four different electro-mechanical switches 47 that allow or prevent the signal from being passed onto one of more of the common data-bus interconnects 48

FIG. 8 shows a method for protecting the solder joints and electrical traces of a surface mounted integrated circuit 51 and connector 53. An annular ring 49 placed over the integrated circuit with curved edges 50 aligned to the base of the circuit minimize bending stresses from being directly applied to the legs and electrical joints 52 of the surface mount device. An annular ring with an opening 55 on one side can be used to minimize bending stresses from being directly applied to the legs and electrical joints 56 of a surface mount electro-mechanical connector 53 attached to a set of electrical interconnections 54.

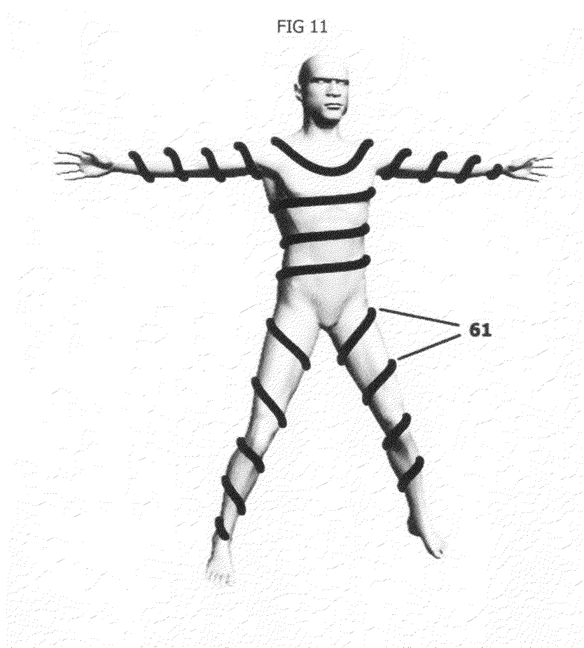

FIG. 9 shows a method for constructing the analog and digital electrical interconnections of a common data-bus 48 with electrical power supplied along the upper two traces, analog signals supplied along the center traces (with alternating ground lines), and digital data and a digital clock signal supplied along the lower traces. Placing the electrical interconnections between an upper layer of electrically conductive and mechanically flexible material 57, and a lower layer of electrically conductive and mechanically flexible material 58, shields the electrical interconnections from electro-magnetic interference (EMI).

FIG. 10 shows the newly proposed wearable electronic system 13 and a method for interconnecting circuits and sensors with pre-cut and pre-shaped lengths and curvatures 59 and 60 that are used to space the modules and sensors at appropriate distances from one another around the human body.

FIG. 11 shows a method for interconnecting circuits and sensors with spiral-like windings 61 to reduce stress on the electrical interconnections caused by bending and/or twisting.

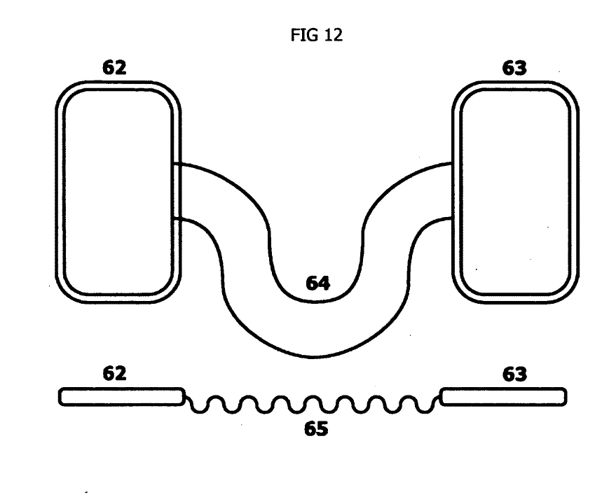

FIG. 12 shows a method for interconnecting circuits and sensors 62 and 63 with serpentine-like shapes 64 or concertina-like shapes 65, to allow for stretching of the interconnects.

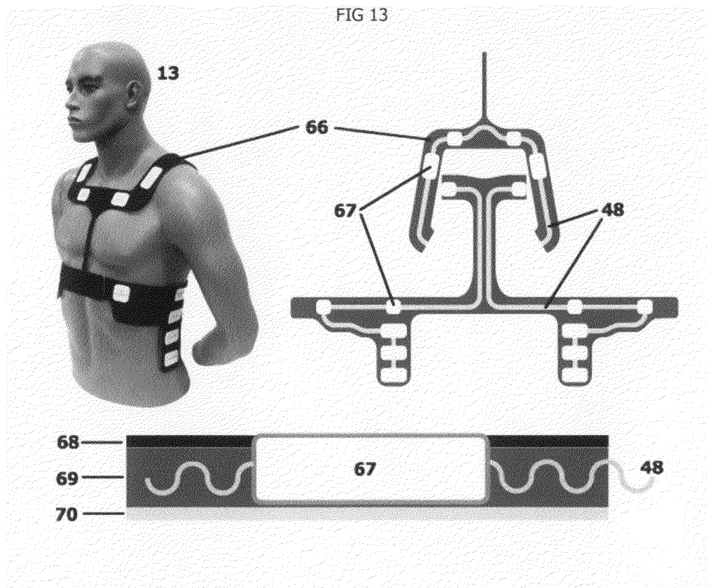

FIG. 13 shows the newly proposed wearable electronic system 13 combined with a three layer cloth-like fabric 66 where openings are created in the cloth garment at strategic locations 67 to allow electronic circuits and sensor modules to be accessed without any removal of the cloth-like fabric. It also shows the electrical data-bus interconnections 48 to be enclosed within the fabric layers 68, 69, and 70.

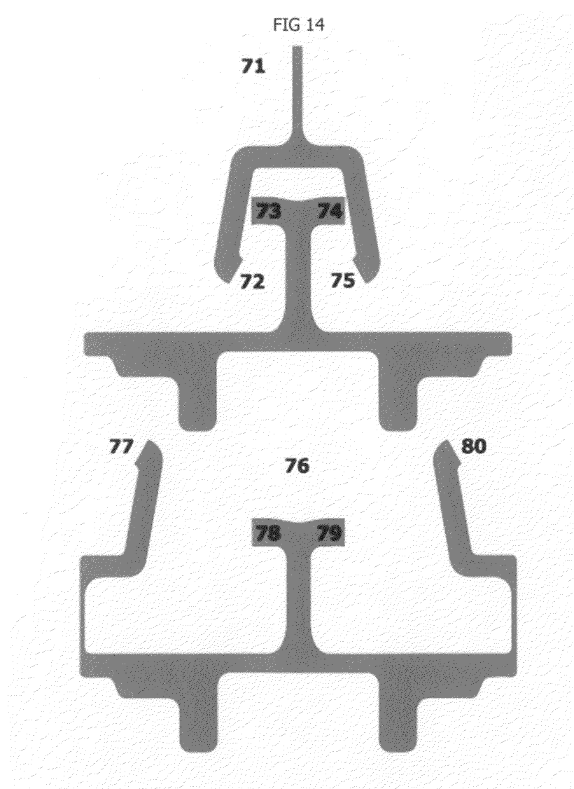

FIG. 14 shows two different garment designs 71 and 76 for donning and doffing over a human torso. Garment 71 is constructed by attaching positions 72 to 73, and 74 to 75 to produce a design similar to that shown in FIG. 13. Garment 76 is constructed by attaching positions 77 to 78, and 79 to 80 to produce a vest or waistcoat like design.

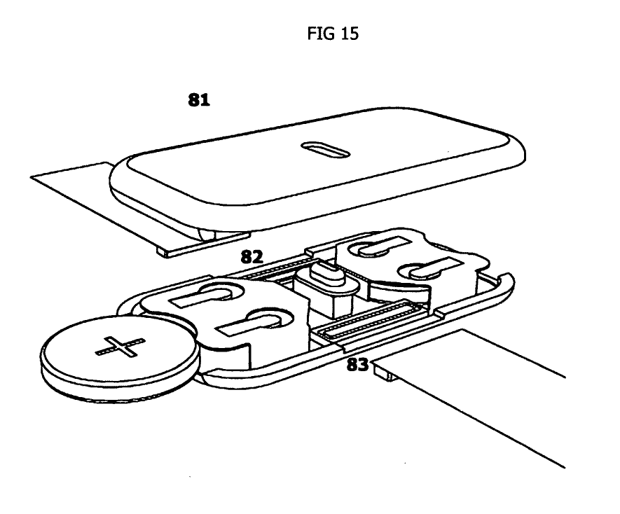

FIG. 15 shows an example of a battery module 81 with dual electro-mechanical connectors 82 and 83 that allows for multiple battery modules to be interconnected within the wearable electronic system.

What is claimed is:

1) electronic circuits and sensor modules fabricated from a flexible, semi-rigid, or rigid type electronic circuit board material that are distributed over different physical locations of a human body or other three dimensional form to distribute bulk and mass; and
multiple electrical interconnections between the said electronic circuits and sensor modules that act as a common data-bus structure that said electronic circuits and sensor modules are connected to; and
said electrical interconnections between the said electronic circuits and sensor modules that are fabricated from electrically conducting materials that can withstand repeated flexing and bending as they are moved, bent, and/or twisted; and
said electrical interconnections between the said electronic circuits and sensor modules to be formed from a flexible circuit board material and/or a series of discrete insulated wires laid flat on a generally, but not necessarily, two-dimensional surface to allow for a low height profile; and
said electrical interconnections between the said electronic circuits and sensor modules such that the flatness, curvature, and flexibility combination may conform to the contours of a human body or other three dimensional form; and
said electrical interconnections between the said electronic circuits and sensor modules such that the flatness, curvature, and flexibility combination reduce rubbing and/or chafing effects on the surface of the human body, or other three dimensional form, that rigid or semi-rigid electrical interconnections induce.
said electrical interconnections between the said electronic circuits and sensors are formed as pre-cut lengths of straight and curved shapes that are used to space said electronic circuits and sensors at appropriate distances from one another around the human body.

2) the system described in claim 1 where a design of mechanical housing for retaining the said electronic circuits and sensor modules can be flat or curved in shape to help conform to any surface the mechanical housing is to be wrapped, draped, bonded, or otherwise attached to or placed on. The mechanical housing construction material may be rigid, semi-rigid, or flexible.

3) the system described in claim 1 where a design of mechanical housing for retaining said electronic circuits and sensor modules has rounded outer edges and corners so as to reduce rubbing and/or chafing effects with any surface the mechanical housing comes into contact with.

4) the system described in claim 1 where a mechanical housing design for retaining said electronic circuits and sensor modules comprises an opening portion of the housing so as to allow access to the electro-mechanical connectors and electrical interconnections of the electronic circuits and/or sensor modules within the mechanical housing.

5) the system described in claim 1 where a mechanical housing design allows for an electrical body sensor (electrode) to snap fit into and out of the housing from an opening hole on one side of the mechanical housing.

6) a wearable electronic system where a mechanical housing design allows for an electrical body sensor (electrode) to snap-fit into and out of the housing and where a physical slider and/or push button mechanism within the housing allows the electrode to be ejected from the housing.

7) the system described in claim 1 where an electrical sampling circuit is placed within a mechanical housing that digitizes the analog physiological signal measured by an electrical body sensor (electrode) connected within the mechanical housing through use of an analog to digital converter (ADC).

8) the system described in claim 1 where an electronic circuit board design allows for a physiological signal measured by a connected electrical body sensor (electrode) to be multiplexed onto any or all of the different analog data-bus electrical interconnections of the electronic circuit board using a suitable electrical or mechanical switch, e.g., an ECG signal switched to electrical traces 1, 2, or 3 etc. as required.

9) the mechanical housing described in claim 2 that uses flexible electrical circuit board to allow a design of electronic circuit that can be curved in shape to help conform to a curved mechanical housing.

10) an annular ring shaped strain relief mechanism with curved and rounded edges for use in protecting the solder joints of electronic components, integrated circuit chips and electrical connectors that are positioned on a flexible electrical circuit board and any flexible electrical traces that are connected to them.

11) the system described in claim 1 where said electronic circuits and sensors include snap-fit or zero-insertion force electro-mechanical connectors to allow for attachment of said electrical interconnections.

12) the system described in claim 1 where said electronic circuits and sensors include pull-force strengthening mechanisms such as, but not limited to, post and hole arrangements and/or retaining clips that mate with the electrical interconnections to prevent them from being accidentally pulled out of their respective electro-mechanical connectors.

13) the system described in claim 1 where said electrical interconnections between the said electronic circuits and sensors use one layer of flexible electrical conductors to transmit and receive analog signals and digital signals on the same layer.

14) the system described in claim 1 where said electrical interconnections between the said electronic circuits and sensors use at least two layers of flexible electrical conductors to transmit and receive analog signals on one layer, and digital signals on a second layer.

15) the system described in claim 1 where said electrical interconnections between the said electronic circuits and sensors use at least one layer of flexible conductive material is overlaid, underlaid, and/or sandwiched in between the said electrical interconnections to shield the analog and digital data-bus signals from electro-magnetic interference (EMI).

16) the system described in claim 1 where said electrical interconnections between the said electronic circuits and sensors and/or the mechanical housings include strain relief mechanisms at any entrance and exit points of the mechanical housing such that the electrical interconnections do not break at the entrance and exit points.

17) the system described in claim 1 where said electrical interconnections between the said electronic circuits and sensors are formed as spiral-like windings to reduce stress on the electrical interconnections caused by bending and/or twisting over a period of time to allow for greater durability of the electrical interconnections.

18) the system described in claim 1 where said electrical interconnections between the said electronic circuits and sensors are formed as serpentine-like shapes to stretch by a greater length than a simple straight line electrical interconnection shape before catastrophic mechanical or electrical failure occurs.

19) the system described in claim 1 where said electrical interconnections between the said electronic circuits and sensors are formed as concertina-like shapes to stretch by a greater length than a simple straight line electrical interconnection shape before catastrophic mechanical or electrical failure occurs.

20) the system described in claim 1 combined with a cloth-like fabric whence the garment is donned and/or doffed and secured and/or released around the torso and over the neck of an existing structure.

21) the system described in claim 1 combined with a cloth-like fabric whence the garment is donned and/or doffed and secured and/or released around the torso of an existing structure.

22) the system described in claim 1 combined with a cloth-like fabric where openings are created in the cloth garment at strategic locations to allow a sensor or electrode to contact with the human skin beneath the opening.

23) a wearable electronic system combined with a cloth-like fabric where openings are created in the cloth garment at strategic locations to allow an electronic circuit and/or sensor module to be accessed without any removal of the cloth-like fabric.

24) a wearable electronic system combined with a cloth-like fabric where the cloth garment is formed from multiple layers of fabric allowing the electrical data-bus interconnections to be enclosed between the fabric layers.

25) the system described in claim 1 where use of electrical body sensors pre-placed at particular positions within a garment automatically locates the electrical body sensors in the correct position relevant to the human body for physiological measurements by donning the garment.

26) the system described in claim 1 where use of a battery pack module with at least two identical electro-mechanical connectors for connecting to at least two other electrical circuits and/or sensor modules.

27) the system described in claim 1 where a software coding system that allows any or all of the electronic circuits and sensors tied to the electrical interconnection to communicate with each other.

28) the system described in claim 1 where flexible, bendable, electrical interconnections electro-mechanically connect to at least one electrical circuit composed of an electrical battery power supply, or a microprocessor unit, or a wireless transceiver, or a memory storage device, or an optical display device, or a microphone, or a three axis accelerometer, or a gas measuring device, or a pulse oxygenation sensor, or a temperature measuring device, or a blood pressure sensor, or a global positioning device, or a respiratory sensor.

29) a wearable electronic system that indicates correct electrical connection between electronic circuits and sensor modules by using light emitting diodes (LEDs) to indicate the presence of electrical signal.
